(19) United States
(12) Patent Application Publication
(10) Pub. No.: US 2025/0347605 A1
(43) Pub. Date: Nov. 13, 2025

(54) CONCENTRATION MEASURING METHOD, CONCENTRATION MEASURING APPARATUS, AND PROGRAM

(71) Applicant: NEC Corporation, Minato-ku, Tokyo (JP)

(72) Inventors: Taichi TANAKA, Tokyo (JP); Toshihiko HIROAKI, Tokyo (JP)

(73) Assignee: NEC Corporation, Minato-ku, Tokyo (JP)

(21) Appl. No.: 18/871,233

(22) PCT Filed: Jun. 8, 2023
(86) PCT No.: PCT/JP2023/021347

§ 371 Date: Dec. 3, 2024

(30) Foreign Application Priority Data
Jun. 23, 2022 (JP) 2022-100972

(51) Int. Cl. G01N 9/18; G01N 33/18

(52) U.S. Cl. CPC G01N 9/18; G01N 33/1886

(57) ABSTRACT

A concentration measuring apparatus of the present invention includes: an acquiring unit that acquires a height position of a float which floats in a liquid and whose floating height changes in accordance with a concentration of the liquid, the height position being measured using a radio wave; and a measuring unit that measures the concentration of the liquid based on the height position.

### TECHNICAL FIELD

The present invention relates to a method for measuring the concentration of a liquid. The present invention also relates to a concentration measuring apparatus, a concentration measuring system, and a program.

### BACKGROUND ART

A method for measuring the salt concentration of salt water in a salt pan is described in Patent Literature 1. First, a method using a hydrometer/Baumé hydrometer is described as a general method for generating a salt water concentration. However, such a method requires collection of salt water samples from a salt pan, and the large area of the salt pan makes it difficult to collect the samples. A problem is described that, as a result, it becomes difficult to frequently monitor the salt water concentration and variations in amounts of salt to be produced become large.

Further, Patent Literature 1 describes, as another salt concentration measuring method, using a new device based on the principle of buoyancy. Specifically, the device has a hemispherical body and a graduated rod placed on the top of the body and, when a desired salt concentration is reached in salt water, floats up to a corresponding scale mark. For this reason, a salt water concentration is measured by observing the scale mark on the device floating in a salt pan from a distance.

### CITATION LIST

### Patent Literature

Patent literature 1: Japanese Translation of PCT International Application Publication No. JP-T 2006-511795

### SUMMARY OF INVENTION

However, the technique described in Patent Literature 1 above causes problems that a salt concentration cannot be measured with high accuracy and that the measurement is time-consuming. Here, many of salt spans have an area of several km², and some have an area of even more. For this reason, in a case where the abovementioned device is floated in a salt pan with such a large area, it is required to observe the scale mark on the device from a distance or to move close to each device to observe. Then, in the case of observing the device from a distance, it is difficult to observe the scale mark with high accuracy. On the other hand, in the case of moving close to each device to observe the scale mark, the movement is time-consuming. As a result, a problem arises that a salt concentration cannot be measured easily or accurately. Moreover, such a problem is not limited to the case of measuring the salt concentration of salt water in a salt pan, and may arise in the case of measuring the concentration of any liquid in any location.

Accordingly, an object of the present disclosure is to provide a method for easily and accurately measure the concentration of a liquid.

### Solution to Problem

A concentration measuring method as an aspect of the present disclosure includes: acquiring a height position of a float measured using a radio wave, the float floating in a liquid, a floating height of the float changing in accordance with a concentration of the liquid; and measuring the concentration of the liquid based on the height position.

Further, a concentration measuring apparatus as an aspect of the present disclosure includes: an acquiring unit that acquires a height position of a float measured using a radio wave, the float floating in a liquid, a floating height of the float changing in accordance with a concentration of the liquid; and a measuring unit that measures the concentration of the liquid based on the height position.

Further, a concentration measuring system as an aspect of the present disclosure includes: a float floating in a liquid, a floating height of the float changing in accordance with a concentration of the liquid; a position measuring apparatus measuring a height position of the float using a radio wave; and a concentration measuring apparatus including an acquiring unit that acquires the measured height position of the float, and a measuring unit that measures the concentration of the liquid based on the height position.

Further, a program as an aspect of the present disclosure includes instructions for causing a computer to execute processes to: acquire a height position of a float measured using a radio wave, the float floating in a liquid, a floating height of the float changing in accordance with a concentration of the liquid; and measure the concentration of the liquid based on the height position.

### Advantageous Effects of Invention

Configured as described above, the present disclosure allows measurement of the concentration of a liquid with ease and with high accuracy.

### BRIEF DESCRIPTION OF DRAWINGS

FIG. 1 is a block diagram showing the overall configuration of a salt concentration measuring system in a first example embodiment of the present disclosure.

FIG. 2 is a view showing an example of the configuration of a float disclosed in FIG. 1.

FIG. 3 is a block diagram showing the configuration of a concentration measuring apparatus disclosed in FIG. 1.

FIG. 4 is a flowchart showing the operation of the concentration measuring apparatus disclosed in FIG. 1.

FIG. 5 is a view showing another example of the configuration of the float disclosed in FIG. 1.

FIG. 6 is a view showing another example of the configuration of the float disclosed in FIG. 1.

FIG. 7 is a block diagram showing the hardware configuration of a concentration measuring apparatus in a second example embodiment of the present disclosure.

FIG. 8 is a block diagram showing the configuration of the concentration measuring apparatus in the second example embodiment of the present disclosure.

### DESCRIPTION OF EXAMPLE EMBODIMENTS

### <First Example Embodiment

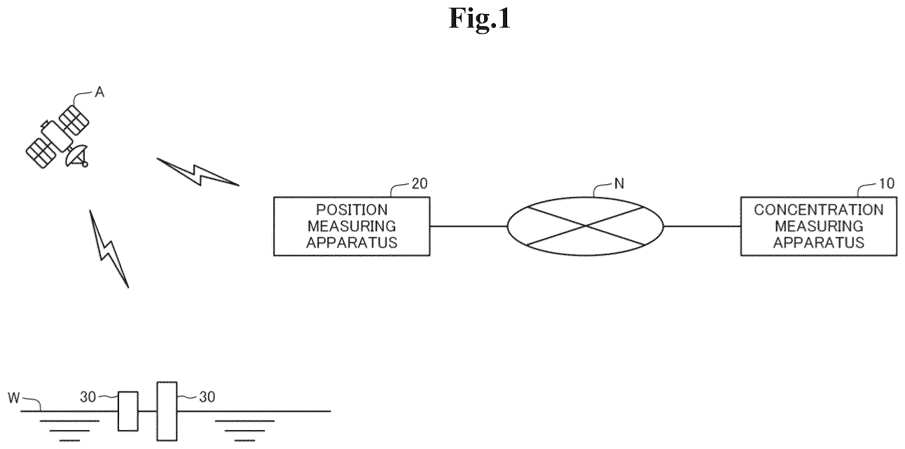

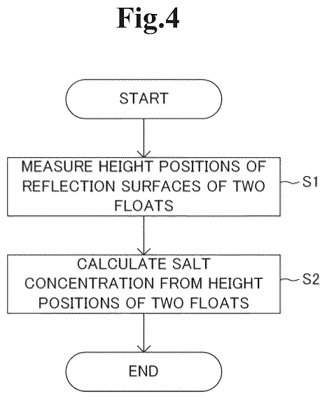

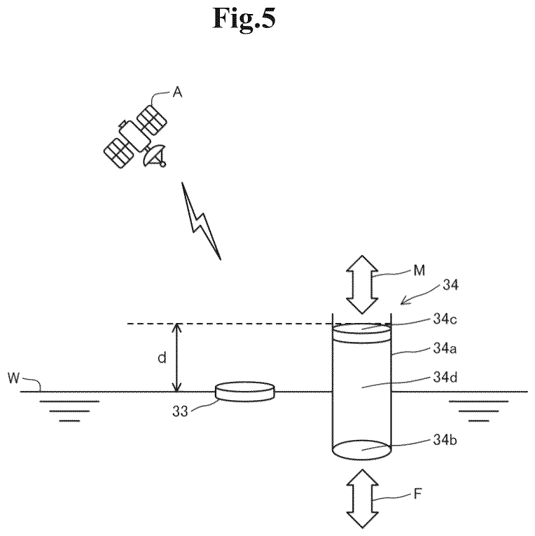

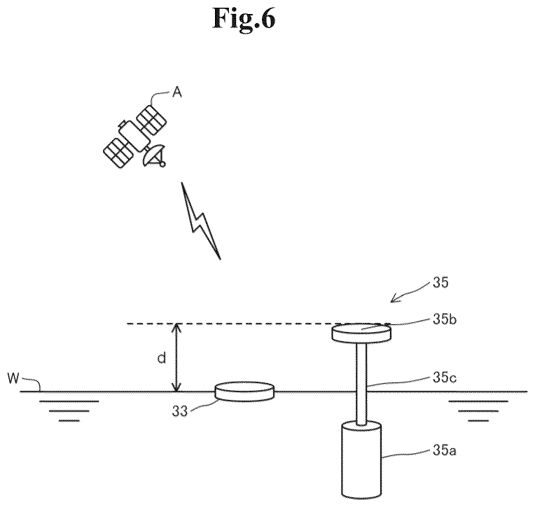

A first example embodiment of the present disclosure will be described with reference to FIGS. 1 to 6. FIGS. 1 to 3 are views for describing the configuration of a salt concentration measuring system, and FIG. 4 is a view for describing the processing operation of the salt concentration measuring system. Moreover, FIGS. 5 and 6 are views for describing other examples of the configuration of the salt concentration measuring system.

The salt concentration measuring system in this example embodiment is for measuring the salt concentration of salt water in a salt pan. In particular, the salt concentration measuring system in this example embodiment is preferred for measuring the salt concentration of salt water in a solar salt pan, where salt-containing water of the sea or a salt lake is evaporated and concentrated using solar heat to produce crystallized salt. That is to say, in the case of a solar salt pan, the system is particularly preferred in a situation where the salt pan has an area of several km² or more. However, the salt concentration measuring system in the present disclosure may be used in a salt pan of any size.

The system of the present disclosure is not limited to being used for measuring the salt concentration of salt water in a salt pan, and can also be applied to measuring the concentration of any liquid present in any location. For example, the system may be applied to measuring the salt concentration of a liquid present in a lake, the sea or the like, or may be applied to measuring the concentration of a colloidal solution such as mud. At this time, a liquid to be a measurement target is, for example, a solution in which a solute is dissolved in a solvent, and the concentration is the ratio of the solute in the solution, but the concentration of any substance dissolved in any liquid may be measured. Moreover, a measurement location may be any location.

As shown in FIG. 1, the salt concentration measuring system in this example embodiment includes a float 30 floating in salt water W of a salt pan, a position measuring apparatus 20 that measures the height position of the float, and a concentration measuring apparatus 10 that measures the salt concentration of the salt water W. Then, in this example embodiment, in order to measure the salt concentration of the salt water W, a synthetic aperture radar (SAR) mounted on an artificial satellite A is used as will be described later. The respective components will be described in detail below.

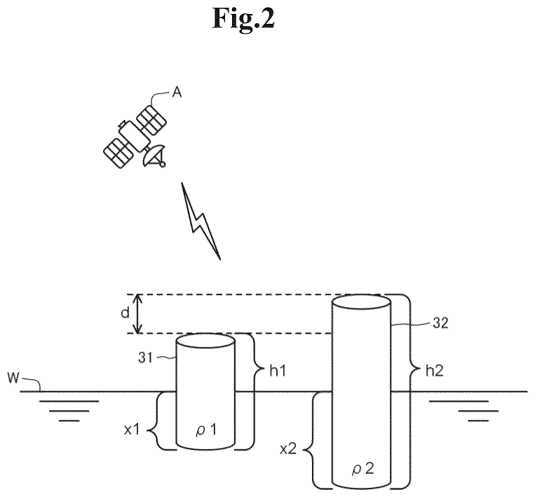

The float 30 is a floating object placed in the salt pan, and is composed of a member having buoyancy that allows it to float in the salt water W in the salt pan. The float 30 is then placed in a plurality of locations in the vast salt pan, for example. Specifically, in this example embodiment, two floats 30 that forms a pair are placed in each location. As an example, as shown in FIG. 2, the float 30 includes two floats forming a pair, that is, a first float 31 and a second float 32, and are arranged in predetermined locations. Here, the first float 31 and the second float 32 are each formed in a cylindrical shape, have the same circular cross-sectional area, and have different heights h1 and h2, with the height h1 of the first float 31 being lower than the height h2 of the second float 32. Then, the upper surfaces of the first float 31 and the second float 32 are each configured as a reflecting part that efficiently reflects radio waves applied by a synthetic aperture radar (hereinafter referred to as “SAR”) mounted on the artificial satellite A as will be described later, in the direction of the SAR. For example, a reflective board serving as the reflecting part may be formed on each of the upper surface of the first float 31 and the second float 32, or a corner reflector may be mounted as the reflecting part on each of the upper parts of the floats. Then, as will be described later, the reflecting part is at the height position of each of the floats to be measured. In addition, as will be described later, the first float 31 and the second float 32 are configured to have different buoyancy amounts corresponding to salt water concentration by making differences in density, overall mass, cross-sectional area, height, and so forth. As a result, the first float 31 and the second float 32 are configured so that the height positions to be measured, which are the positions of the reflecting parts, are different from each other for the salt water W having the same salt water concentration.

Here, the first float 31 and the second float 32 are arranged so as to float in the salt water W with the height directions of the cylindrical shapes positioned in the vertical direction. For example, the first float 31 and the second float 32 are arranged in the salt water W in a state in which they are contained in a predetermined frame or connected by a predetermined string or rod, and the postures of the floats are thereby determined. At this time, the first float 31 and the second float 32 are preferably arranged along the direction of application of the radio waves by the SAR. That is to say, the first float 31 and the second float 32 are preferably arranged along a direction perpendicular to the direction of travel of the artificial satellite A on which the SAR is mounted. Also in this case, the arrangement is restricted by a predetermined frame, string, rod, or the like, as described above.

Furthermore, the first float 31 and the second float 32 are configured to have different densities ρ1 and ρ2 and, for example, configured so that the density ρ1 of the first float 31 is greater than the density ρ2 of the second float 32. For this reason, as shown in FIG. 2, the first float 31 and the second float 32 have different sinking volumes in the same salt concentration of salt water W, and the first float 31 sinks to a shorter height than the second float 32. Then, the height positions of the upper parts of the first float 31 and the second float 32, that is, the height positions of the radio wave reflecting parts are different in the same salt concentration of salt water W, and a difference d therebetween corresponds to the salt concentration of the salt water W. In other words, when the salt concentration of the salt water W changes, the height positions of the first float 31 and the second float 32 change, and the difference d therebetween also changes. In addition, the density of each of the first float 31 and the second float 32 does not need to be uniform throughout the entirety of the float. For example, by making the density of the lower end side of the float higher, the floating attitude of the float can be stabilized. However, the first float 31 and the second float 32 may have the same density, in which case they are preferably formed with different sizes of cross-sectional shapes. Consequently, sinking heights, that is, floating amounts differ for the same salt concentration of salt water W, the height positions of the radio wave reflecting parts also differ, and the difference d therebetween corresponds to the salt concentration of the salt water W. In other words, the abovementioned difference d is obtained by eliminating the influence of a height change dependent on a factor other than the concentration of the salt water W. For example, the water level of the salt water W in the salt pan changes, and the change of the water level is directly reflected as a change of the heights of both the floats; however, by obtaining the difference d between the height positions of the floats, the change of the water level of the salt water W can be eliminated. As a result, it becomes possible to measure a salt concentration with higher accuracy using the difference d as will be described later.

The artificial satellite A is called an SAR satellite and is equipped with a synthetic aperture radar (SAR). An SAR can measure a distance to a target object to be observed by applying radio waves onto the ground and observing the radio waves reflected from the target object. Then, the artificial satellite A in this example embodiment targets the float 30 floating in the salt water W of the salt pan, and transmits information on the radio waves applied to and observed from the float 30 to the position measuring apparatus 20. At this time, in a case where data from two or more SARs are used, a change in distance to the target object can be measured with high accuracy using a technique called interferometric SAR, for example. For example, in a case where the data from the two or more SARs are acquired in different orbits, height can be acquired as a result of analyzing a change in distance due to the different orbits. Moreover, in a case where the data from the two or more SARs are acquired on different dates, a change in height can be acquired with high accuracy based on a change in distance between the dates, and the height position itself can be acquired with high accuracy by accumulating the changes in height. At this time, by actually measuring and storing the initial positions of the respective floats in advance, and multiplying the actual measurement values by the height change values of the respective floats as described above, it is possible to calculate the heights of the respective floats and calculate the difference d therebetween.

The position measuring apparatus 20 is configured with one or a plurality of information processing apparatuses each including an arithmetic logic unit and a memory unit. The position measuring apparatus 20 receives information on the radio waves acquired from the artificial satellite A as described above, and measures the height position of the float 30 from the information on the radio waves, for example, by the abovementioned method. In particular, in this example embodiment, the position measuring apparatus 20 measures the respective height positions of the first float 31 and the second float 32 forming a pair, which are placed at the predetermined locations as described above, and also measures the difference d between the height positions. At this time, the position measuring apparatus 20 previously stores the position information of each pair of floats 30 placed at a plurality of locations within the salt pan, and based on the information on the radio waves including position information from the SAR, measures the height positions of the floats 30 forming a pair placed at each of the locations and the difference d therebetween. Then, the position measuring apparatus 20 transmits the measured height positions and difference d together with the identification information of the floats located at each of the locations to the concentration measuring apparatus 10 connected via the network N. In addition, the position measuring apparatus 20 may transmit only the measured height positions to the concentration measuring apparatus 10, and may not calculate the difference therebetween or transmit to the concentration measuring apparatus 10.

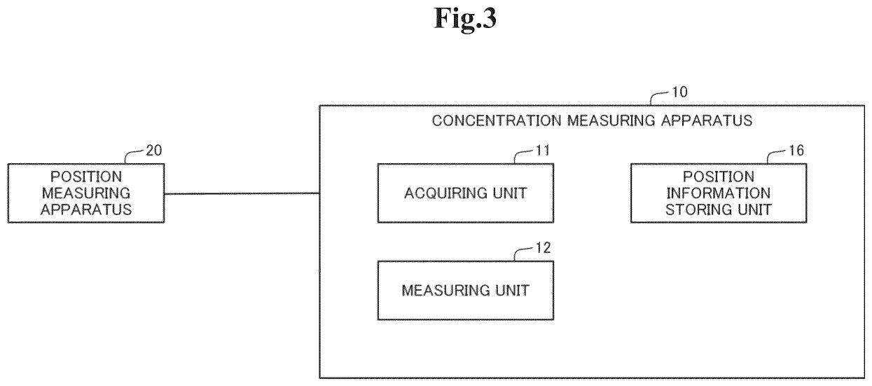

The concentration measuring apparatus 10 (salt concentration measuring apparatus) is configured with one or a plurality of information processing apparatuses each including an arithmetic logic unit and a memory unit. The concentration measuring apparatus 10 is, for example, an information processing apparatus managed by a business operator that manages the salt concentration of the salt water W in the salt pan. Then, as shown in FIG. 3, the concentration measuring apparatus 10 includes an acquiring unit 11 and a measuring unit 12. The respective functions of the acquiring unit 11 and the measuring unit 12 can be realized by the arithmetic logic unit executing a program for realizing the respective functions that is stored in the memory unit. The concentration measuring apparatus 10 also includes a position information storing unit 16. The position information storing unit 16 is configured with the memory unit. Each of the components and the operation thereof will be described in detail below.

The acquiring unit 11 acquires float height position information transmitted from the position measuring apparatus 20 (step S1 in FIG. 4) and stores the information into the position information storing unit 16. At this time, the acquiring unit 11 stores identification information corresponding to the float placed at each of the locations in association with the transmitted height position information of the floats forming a pair. For example, in the example of FIG. 2, the acquiring unit 11 acquires and stores identification information corresponding to the float at a predetermined location and also the height positions of the first float 31 and the second float 32. In a case where, in addition to the height position information of the first float 31 and the second float 32, information on the difference d therebetween is also transmitted from the position measuring apparatus 20, the acquiring unit 11 may also acquire and store the information on the difference d.

The measuring unit 12 measures the salt concentration of the salt water W in which the float 30 floats based on the acquired height position of the float 30 (step S2 in FIG. 4). In this example embodiment, the measuring unit 12 measures the salt concentration of the salt water W based on the difference d between the height positions of the first float 31 and the second float 32 forming a pair. Here, in the example shown in FIG. 2, as described above, the first float 31 and the second float 32 are each formed in a cylindrical shape, have the same circular cross-sectional area, and have different heights h1 and h2. Then, since the first float 31 and the second float 32 are configured to have different densities ρ1 and ρ2, heights x1 and x2 to which they sink in the salt water W are different, resulting in the difference d in height positions. In this case, the salt concentration p of the salt water W can be found, for example, by Expression 1 shown below.

$$ρ = \frac{ρ 1 h 1 - ρ 2 h 2}{h 1 - h 2 + d} \qquad [ Expression 1 ]$$

The measuring unit 12 measures the salt concentration p as described above for each location where the float 30 is placed based on the height position information of the float 30 associated with the location. Then, the measuring unit 12 stores the measured salt concentration p into the position information storing unit 16 in association with the location where the float is placed. The measuring unit 12 may output the measured salt concentration p together with information on the corresponding location, or may compare it with a preset threshold value or a value measured thereafter and output the comparison result.

As described above, in the salt concentration measuring system of this example embodiment, it is possible to measure the salt concentration of the salt water in the salt pan by measuring the height position of the float 30 using the SAR mounted on the artificial satellite A. Consequently, it is possible to measure the salt concentration with ease and with high accuracy, even in a large salt pan.

Although a case of measuring the height position of the float 30 using the SAR mounted on the artificial satellite A has been illustrated above, the SAR is not necessarily limited to being mounted on the artificial satellite A, and may be mounted on an aircraft or may be mounted on another flying object or a structure. Moreover, the height position of the float 30 is not necessarily limited to being measured using the SAR, and may be measured by any method using radio waves emitted from the sky above the salt pan. Alternatively, only when the weather is good, the THz band or the optical frequency band may be used as electromagnetic waves. That is to say, high-frequency electromagnetic waves are inferior in terms of straightness and transparency, but they can be used under conditions where there are no obstructions. Moreover, the concentration measuring apparatus 10 is not necessarily limited to acquiring the height position of the float 30 measured by the position measuring apparatus 20, and may acquire the height position of the float 30 directly from a device such as a SAR, or may acquire the position from any device.

Here, a modified example of the configuration of the salt concentration measuring system configured as described above, particularly, a modified example of the float 30 will be described with reference to FIGS. 5 and 6.

As shown in FIG. 5, the float 30 in the modified example of this example embodiment may include a pair of a third float 33 and a fourth float 34. The third float 33 is formed of, for example, a highly corrosion-resistant metal thin film member and is configured to, for example, float on the water surface regardless of the salt concentration of the salt water W, and the upper surface thereof is configured as a reflecting part. The fourth float 34 is configured so that its floating height changes in accordance with the osmotic pressure of the salt water W. Specifically, the fourth float 34 includes a hollow cylindrical body 34a, an osmosis membrane 34b that covers the lower part of the cylindrical body 34a and is located in the salt water W, a reflecting part 34c that covers the upper part of the cylindrical body 34a, and a contained liquid 34d such as water that is contained within the cylindrical body 34a, that is, between the osmosis membrane 34b and the reflecting part 34c. Consequently, an osmotic pressure F is applied vertically to the osmotic membrane 34b in accordance with a change in the salt concentration of the salt water W, and in accordance with this, the reflecting part 34c on the upper surface moves in a vertical direction M, so that the height position changes. Thus, the third float 33 and the fourth float 34 have measured height positions different from each other that are the positions of the reflecting parts, for the salt water W with the same salt water concentration, and the difference d between the height positions of the third float 33 and the fourth float 34 changes in accordance with change of the salt concentration.

In such a configuration, the artificial satellite A can measure the height positions of the third float 33 and the fourth float 34 using the SAR, and the concentration measuring apparatus 10 can acquire them. The concentration measuring apparatus 10 can then calculate the density p of the salt water W using the osmotic pressure and so forth based on the difference d between the acquired height positions of the third float 33 and the fourth float 34. At this time, the concentration measuring apparatus 10 may, for example, prepare a correspondence relation between the difference d between the height positions of floats forming a pair and the salt concentration p of the salt water W, and measure the value of the salt concentration p corresponding to the difference d between the measured height positions of the floats as the salt concentration at the time of measurement.

Further, as shown in FIG. 6, the float 30 in another modified example of this example embodiment may include a pair of a third float 33 and a fifth float 35. The third float 33 is the same as that shown in FIG. 5 described above and is configured to, for example, float on the water surface of the salt water W regardless of the salt concentration thereof, and the upper surface thereof is configured as a reflecting part. The fifth float 35 includes a hydrometer part 35a that has a predetermined density and is placed within the salt water W and whose height position within the salt water W changes in accordance with the salt concentration, and a reflecting part 35c that is connected to the hydrometer part 35a via a rod-shaped body 35b and is located at the top. Consequently, the hydrometer part 35a is displaced vertically in response to change of the salt concentration of the salt water W and, in response to this, the reflecting part 35c at the top can move vertically, so that the height position changes. Thus, the third float 33 and the fifth float 35 have measured height positions different from each other that are the positions of the reflecting parts, for the salt water W with the same salt water concentration, and the difference d between the height positions of the third float 33 and the fifth float 35 changes in accordance with change of the salt concentration.

With such a configuration, the artificial satellite A can measure the height positions of the third float 33 and the fifth float 35 using the SAR, and the concentration measuring apparatus 10 can acquire them. The concentration measuring apparatus 10 can then calculate the density p of the salt water W based on the difference d between the acquired height positions of the third float 33 and the fifth float 35 using the density of the hydrometer part 35a, and so forth. At this time, the concentration measuring apparatus 10 may, for example, prepare a correspondence relation between the difference d between the height positions of floats forming a pair and the salt concentration p of the salt water W, and measure the value of the salt concentration p corresponding to the difference d between the measured height positions of the floats as the salt concentration at the time of measurement.

In the above, a case has been illustrated in which the float 30 placed at the location where the salt concentration is measured includes two floats forming a pair, but the float 30 may be formed of a single float. For example, it is possible to measure the height position of the single float 30 as described above using radio waves and also measure the height position of the water surface of the salt water W in the salt pan, and thereby measure the salt concentration of the salt water W using the difference between the height position of the water surface of the salt water W and the height position of the float 30. The height of the water surface can also be obtained simply by measuring the reflection from the water surface, but the reflection of the radio waves may also be strengthened by floating particles, a film or the like on the water surface. Alternatively, the water surface may be measured using, among measurement methods using radio waves, a measurement method of acquiring specular reflection of the radio waves with sensitivity by mounting a receiver and a transmitter on different flying objects or the like. Alternatively, by using the height of the land instead of the height position of the water surface of the salt water W, the difference between the height positions of the land and the float 30 may be measured. Moreover, since the water surface of the salt pan becomes specular surface, it is possible to use the mirror image of the reflecting part of the float 30 and utilize the interference thereof to calculate the salt concentration. It is also possible to calculate the salt concentration using the difference between the height position of the float 30 and the height position of another reference object or point that is not displaced.

### Second Example Embodiment

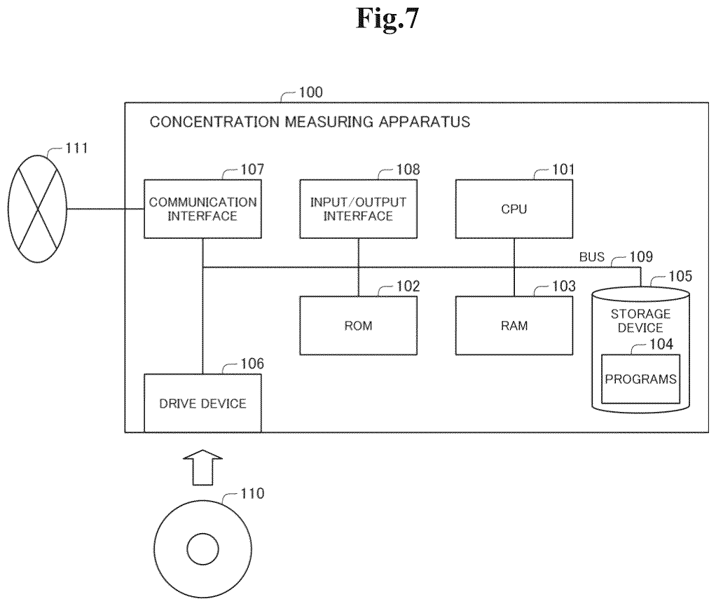

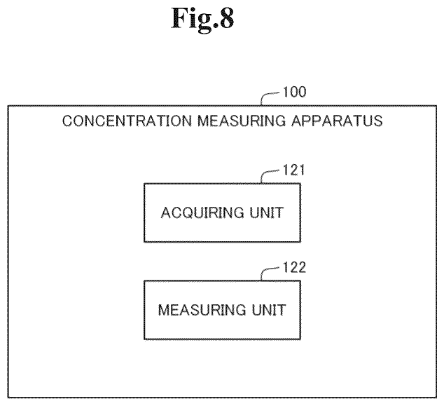

Next, a second example embodiment of the present disclosure will be described with reference to FIGS. 7 and 8. FIGS. 7 and 8 are block diagrams showing the configuration of a concentration measuring apparatus in the second example embodiment. In this example embodiment, the overview of the configuration of the concentration measuring apparatus described in the above example embodiment is shown.

First, the hardware configuration of a concentration measuring apparatus 100 in this example embodiment will be described with reference to FIG. 7. The concentration measuring apparatus 100 is configured with a general information processing apparatus and, as an example, has the following hardware configuration including:
a CPU (Central Processing Unit) 101 (arithmetic logic unit);
a ROM (Read Only Memory) 102 (memory unit);
a RAM (Random Access Memory) 103 (memory unit);
programs 104 loaded to the RAM 103;
a storage device 105 storing the programs;
a drive device 106 that reads from and writes into a storage medium 110 outside the information processing apparatus;
a communication interface 107 connected to a communication network 111 outside the information processing apparatus;
an input/output interface 108 that inputs and outputs data; and
a bus 109 that connects the components.

Then, the concentration measuring apparatus 100 can structure and include an acquiring unit 121 and a measuring unit 122 shown in FIG. 8 by acquisition and execution of the programs 104 by the CPU 101. The programs 104 are, for example, stored in advance in the storage device 105 or the ROM 102, and loaded to the RAM 103 and executed by the CPU 101 as necessary. In addition, the programs 104 may be provided to the CPU 101 via the communication network 111, or the programs may be stored in the storage medium 110 in advance and read out by the drive device 106 and provided to the CPU 101. However, the acquiring unit 121 and the measuring unit 122 mentioned above may be structured with dedicated electronic wires for realizing such means.

FIG. 7 shows an example of the hardware configuration of the information processing apparatus serving as the concentration measuring apparatus 100, and the hardware configuration of the information processing apparatus is not limited to the abovementioned case. For example, the information processing apparatus may include part of the abovementioned configuration, such as not having the drive device 106. Moreover, the information processing apparatus can use a GPU (Graphic Processing Unit), a DSP (Digital Signal Processor), an MPU (Micro Processing Unit), an FPU (Floating point number Processing Unit), a PPU (Physics Processing Unit), a TPU (Tensor Processing Unit), a quantum processor, a microcontroller or a combination thereof, instead of the abovementioned CPU.

The abovementioned acquiring unit 121 acquires the height position of a float which floats in a liquid and whose floating height changes in accordance with the concentration of the liquid. At this time, the acquired height position of the float is measured using radio waves, for example, measured by emission of radio waves from the sky. For example, the float includes two floats whose measured height positions are different in a liquid with the same concentration, and the difference between the measured height positions changes in accordance with the concentration, and the acquiring unit 121 acquires the height positions of these two floats.

The abovementioned measuring unit 122 measures the concentration of the liquid based on the height positions. At this time, the float changes its measured height position in accordance with the concentration of the liquid, so that it is possible to measure the concentration of the liquid in which the float is floating, using the height positions. In particular, it is possible to measure the concentration using the difference between the height positions of the two floats as described above.

Configured as described above, the present disclosure allows measurement of the height position of a float using radio waves to measure the concentration of a liquid. Therefore, even if the liquid is present in a vast region, it is possible to measure the concentration with ease and with high accuracy.

In addition, the abovementioned program can be stored using various types of non-transitory computer-readable mediums and provided to a computer. Non-transitory computer-readable mediums include various types of tangible storage mediums. Examples of non-transitory computer-readable mediums include a magnetic recording medium (e.g., a flexible disk, a magnetic tape, a hard disk drive), a magneto-optical recording medium (e.g., a magneto-optical disk), a CD-ROM (Read Only Memory), a CD-R, a CD-R/W, and a semiconductor memory (e.g., a mask ROM, a PROM (Programmable ROM), an EPROM (Erasable PROM), a flash ROM, a RAM (Random Access Memory)). The program may also be provided to the computer by various types of transitory computer-readable mediums. Examples of transitory computer-readable mediums include electrical signals, optical signals, and electromagnetic waves. The transitory computer-readable medium can provide the program to the computer via a wired communication path, such as an electric wire or an optical fiber, or via a wireless communication path.

Although the present disclosure has been described above with reference to the above example embodiments, the present disclosure is not limited to the above example embodiments. The configurations and details of the present disclosure can be changed in various manners that can be understood by one skilled in the art within the scope of the present disclosure. Moreover, at least one or more of the functions of the acquiring unit 121 and the measuring unit 122 described above may be executed by an information processing apparatus installed in anywhere on the network and connected, that is, may be executed by so-called cloud-computing.

### <Supplementary Notes>

The whole or part of the example embodiments disclosed above can be described as the following supplementary notes. Below, the overview of the configurations of a concentration measuring method, a concentration measuring apparatus and a program according to the present invention will be described. However, the present invention is not limited to the following configurations.

### (Supplementary Note 1)

A concentration measuring method, comprising:
acquiring a height position of a float measured using a radio wave, the float floating in a liquid, a floating height of the float changing in accordance with a concentration of the liquid; and
measuring the concentration of the liquid based on the height position.

### (Supplementary Note 2)

The concentration measuring method according to Supplementary Note 1, comprising:
acquiring the respective height positions of the two floats floating so that the height positions to be measured are different in the liquid with the same concentration; and
measuring the concentration of the liquid based on the height positions of the two floats.

### (Supplementary Note 3)

The concentration measuring method according to Supplementary Note 2, comprising:
acquiring the respective height positions of the two floats floating so that a difference between the height positions to be measured changes in accordance with the concentration of the liquid; and
measuring the concentration of the liquid based on the difference between the height positions of the two floats.

### (Supplementary Note 4)

The concentration measuring method according to Supplementary Note 2 or 3, comprising
acquiring the respective height positions of the two floats having different densities.

### (Supplementary Note 5)

The concentration measuring method according to Supplementary Note 2 or 3, comprising
acquiring the height position of at least one of the floats that floats so that the height position to be measured changes in accordance with an osmotic pressure of the liquid.

### (Supplementary Note 6)

The concentration measuring method according to any of Supplementary Notes 2 to 5, comprising
acquiring the respective height positions of the two floats, one of the floats floating with the height position being a position of a water surface of the liquid, another of the floats floating with the height position being above the position of the water surface of the liquid.

### (Supplementary Note 7)

The concentration measuring method according to any of Supplementary Notes 1 to 6, comprising
acquiring the height position of the float measured using a synthetic aperture radar mounted on a flying object.

### (Supplementary Note 8)

The concentration measuring method according to Supplementary Note 7, wherein
a reflecting part is formed at a top of the float, the reflecting part reflecting the radio wave applied by the synthetic aperture radar in a direction of the synthetic aperture radar.

### (Supplementary Note 9)

The concentration measuring method according to Supplementary Note 7 or 8, comprising
acquiring the height positions of the two floats placed along a direction in which the radio wave is applied by the synthetic aperture radar.

### (Supplementary Note 10)

A concentration measuring apparatus, comprising:
an acquiring unit that acquires a height position of a float measured using a radio wave, the float floating in a liquid, a floating height of the float changing in accordance with a concentration of the liquid; and
a measuring unit that measures the concentration of the liquid based on the height position.

### (Supplementary Note 10.1)

The concentration measuring apparatus according to Supplementary Note 10, wherein:
the acquiring unit acquires the respective height positions of the two floats floating so that the height positions to be measured are different in the liquid with the same concentration; and
measuring the concentration of the liquid based on the height positions of the two floats.

### (Supplementary Note 10.2)

The concentration measuring apparatus according to Supplementary Note 10.1, wherein:
the acquiring unit acquires the respective height positions of the two floats floating so that a difference between the height positions to be measured changes in accordance with the concentration of the liquid; and
the measuring unit measures the concentration of the liquid based on the difference between the height positions of the two floats.

### (Supplementary Note 10.3)

The concentration measuring apparatus according to Supplementary Note 10.1 or 10.2, wherein
the acquiring unit acquires the respective height positions of the two floats having different densities.

### (Supplementary Note 10.4)

The concentration measuring apparatus according to Supplementary Note 10.1 or 10.2, wherein
the acquiring unit acquires the height position of at least one of the floats that floats so that the height position to be measured changes in accordance with an osmotic pressure of the liquid.

### (Supplementary Note 10.5)

The concentration measuring apparatus according to any of Supplementary Notes 10.1 to 10.4, wherein
the acquiring unit acquires the respective height positions of the two floats, one of the floats floating with the height position being a position of a water surface of the liquid, another of the floats floating with the height position being above the position of the water surface of the liquid.

### (Supplementary Note 11)

A concentration measuring system, comprising:
a float floating in a liquid, a floating height of the float changing in accordance with a concentration of the liquid;
a position measuring apparatus measuring a height position of the float using a radio wave; and
a concentration measuring apparatus including: an acquiring unit that acquires the measured height position of the float; and a measuring unit that measures the concentration of the liquid based on the height position.

### (Supplementary Note 11.1)

The concentration measuring system according to Supplementary Note 11, wherein:
the float includes the two floats floating so that the height positions to be measured are different in the liquid with the same concentration;
the position measuring apparatus measures the respective height positions of the two floats;
the acquiring unit of the concentration measuring apparatus acquires the respective height positions of the two floats; and
the measuring unit of the concentration measuring apparatus measures the concentration of the liquid based on the height positions of the two floats.

### (Supplementary Note 11.2)

The concentration measuring system according to Supplementary Note 11.1, wherein:
the float includes the two floats that float so that a difference between the height positions to be measured changes in accordance with the concentration of the liquid;
the position measuring apparatus measures the respective height positions of the two floats;
the acquiring unit of the concentration measuring apparatus acquires the respective height positions of the two floats; and
the measuring unit of the concentration measuring apparatus measures the concentration of the liquid based on the difference between the height positions of the two floats.

### (Supplementary Note 11.3)

The concentration measuring system according to Supplementary Note 11.1 or 11.2, wherein
the float includes the two floats having different densities.

### (Supplementary Note 11.4)

The concentration measuring system according to Supplementary Note 11.1 or 11.2, wherein
the float includes the two floats, at least one of which floats so that the height position to be measured changes in accordance with an osmotic pressure of the liquid.

### (Supplementary Note 11.5)

The concentration measuring system according to any of Supplementary Notes 11.1 to 11.4, wherein
the float includes the two floats, one of the floats floating so that the height position is a position of a water surface of the liquid, another of the floats floating so that the height position is above the position of the water surface of the liquid.

### (Supplementary Note 11.6)

The concentration measuring system according to any of Supplementary Notes 11 to 11.5, wherein
the position measuring apparatus measures the height position of the float using information measured using a synthetic aperture radar mounted on a flying object.

### (Supplementary Note 11.7)

The concentration measuring system according to Supplementary Note 11.6, wherein
the float includes a reflecting part formed at a top thereof, the reflecting part reflecting the radio wave applied by the synthetic aperture radar in a direction of the synthetic aperture radar.

### (Supplementary Note 11.8)

The concentration measuring system according to Supplementary Note 11.6 or 11.7, wherein
the float includes the two floats placed along a direction in which the radio wave is applied by the synthetic aperture radar.

### (Supplementary Note 20)

A program comprising instructions for causing a computer to execute processes to:
acquire a height position of a float measured using a radio wave, the float floating in a liquid, a floating height of the float changing in accordance with a concentration of the liquid; and
measure the concentration of the liquid based on the height position.

The present invention is based upon and claims the benefit of priority from Japanese patent application No. 2022-100972, filed on Jun. 23, 2022, the disclosure of which is incorporated herein in its entirety by reference.

### REFERENCE SIGNS LIST

10 concentration measuring apparatus
11 acquiring unit
12 measuring unit
16 position information storing unit
20 position measuring apparatus
30, 31, 32, 33, 34, 35 float
A artificial satellite
W salt water
100 concentration measuring apparatus
101 CPU
102 ROM
103 RAM
104 programs
105 storage device
106 drive device
107 communication interface
108 input/output interface
109 bus
110 storage medium
111 communication network
121 acquiring unit
122 measuring unit

What is claimed is:

1. A concentration measuring method, comprising:
acquiring a height position of a float measured using a radio wave, the float floating in a liquid, a floating height of the float changing in accordance with a concentration of the liquid; and
measuring the concentration of the liquid based on the height position.

2. The concentration measuring method according to claim 1, comprising:
acquiring the respective height positions of the two floats floating so that the height positions to be measured are different in the liquid with the same concentration; and
measuring the concentration of the liquid based on the height positions of the two floats.

3. The concentration measuring method according to claim 2, comprising:
acquiring the respective height positions of the two floats floating so that a difference between the height positions to be measured changes in accordance with the concentration of the liquid; and
measuring the concentration of the liquid based on the difference between the height positions of the two floats.

4. The concentration measuring method according to claim 2, comprising
acquiring the respective height positions of the two floats having different densities.

5. The concentration measuring method according to claim 2, comprising
acquiring the height position of at least one of the floats that floats so that the height position to be measured changes in accordance with an osmotic pressure of the liquid.

6. The concentration measuring method according to claim 2, comprising
acquiring the respective height positions of the two floats, one of the floats floating with the height position being a position of a water surface of the liquid, another of the floats floating with the height position being above the position of the water surface of the liquid.

7. The concentration measuring method according to claim 1, comprising
acquiring the height position of the float measured using a synthetic aperture radar mounted on a flying object.

8. The concentration measuring method according to claim 7, wherein
a reflecting part is formed at a top of the float, the reflecting part reflecting the radio wave applied by the synthetic aperture radar in a direction of the synthetic aperture radar.

9. The concentration measuring method according to claim 7, comprising
acquiring the height positions of the two floats placed along a direction in which the radio wave is applied by the synthetic aperture radar.

10. A concentration measuring apparatus, comprising:
at least one memory storing processing instructions; and
at least one processor configured to execute the processing instructions to:
acquire a height position of a float measured using a radio wave, the float floating in a liquid, a floating height of the float changing in accordance with a concentration of the liquid; and
measure the concentration of the liquid based on the height position.

11. A concentration measuring system, comprising:
a float floating in a liquid, a floating height of the float changing in accordance with a concentration of the liquid;
a position measuring apparatus measuring a height position of the float using a radio wave; and
a concentration measuring apparatus acquiring the measured height position of the float, and measuring the concentration of the liquid based on the height position.

12. The concentration measuring system according to claim 11, wherein:
the float includes the two floats floating so that the height positions to be measured are different in the liquid with the same concentration;
the position measuring apparatus measures the respective height positions of the two floats; and
the concentration measuring apparatus acquires the respective height positions of the two floats; and
measures the concentration of the liquid based on the height positions of the two floats.

13. The concentration measuring system according to claim 12, wherein:
the float includes the two floats that float so that a difference between the height positions to be measured changes in accordance with the concentration of the liquid;
the position measuring apparatus measures the respective height positions of the two floats; and
the concentration measuring apparatus acquires the respective height positions of the two floats; and
measures the concentration of the liquid based on the difference between the height positions of the two floats.

14. The concentration measuring system according to claim 12, wherein
the float includes the two floats having different densities.

15. The concentration measuring system according to claim 12, wherein
the float includes the two floats, at least one of which floats so that the height position to be measured changes in accordance with an osmotic pressure of the liquid.

16. The concentration measuring system according to claim 12, wherein
the float includes the two floats, one of the floats floating so that the height position is a position of a water surface of the liquid, another of the floats floating so that the height position is above the position of the water surface of the liquid.

17. The concentration measuring system according to claim 11, wherein
the position measuring apparatus measures the height position of the float using information measured using a synthetic aperture radar mounted on a flying object.

18. The concentration measuring system according to claim 17, wherein
the float includes a reflecting part formed at a top thereof, the reflecting part reflecting the radio wave applied by the synthetic aperture radar in a direction of the synthetic aperture radar.

19. The concentration measuring system according to claim 17, wherein
the float includes the two floats placed along a direction in which the radio wave is applied by the synthetic aperture radar.

20. (canceled)
